(19) United States
(12) Patent Application Publication
(10) Pub. No.: US 2022/0370173 A1
(43) Pub. Date: Nov. 24, 2022

(54) Multi-function Oral Appliance

(71) Applicant: Americo Fernandes, Winnipeg (CA)

(72) Inventor: Americo Fernandes, Winnipeg (CA)

(21) Appl. No.: 17/876,759

(22) Filed: Jul. 29, 2022

Related U.S. Application Data
(63) Continuation of application No. PCT/IB2021/050694, filed on Jan. 29, 2021.
(60) Provisional application No. 63/135,220, filed on Jan. 8, 2021.
(60) Provisional application No. 62/975,528, filed on Feb. 12, 2020.

(51) Int. Cl. A61C 7/08; A61C 9/00; A61M 16/08

(52) U.S. Cl. CPC A61C 7/08; A61C 9/0006; A61M 16/0875

(57) ABSTRACT

Example embodiments of the present invention provide a multi-function oral appliance, comprising an elastomeric polymer, a hard non-elastic polymer, or a combination of the two fused together; formed using 3D printing or vacuum-forming; configured to engage all or part of the bottom and top teeth such that the lower jaw is advanced, e.g., 8 mm, anterior to its habitual resting position and opened, e.g., 8 mm, from its habitual resting position when the appliance is applied; and configured such that, e.g., 90%, or more, of airflow through the mouth is blocked by the appliance. In some embodiments, the appliance is configured to include gel-wells in the interior of the appliance.

### TECHNICAL FIELD

The subject invention relates to oral appliances, specifically oral appliances that provide multiple functions.

### BACKGROUND ART

Oral appliances are used for a variety of purposes, including as helps to alleviate snoring, as guards to protect teeth against night-time grinding, and as delivery mechanisms for various therapies such as medicinal gels. Conventional appliances provide only one function due to specific needs of each function that generally preclude service for another function. There is a need for an oral appliance that can perform several functions in a single appliance.

### DISCLOSURE OF INVENTION

Example embodiments of the present invention provide a multi-function oral appliance, comprising an elastomeric polymer, a hard non-elastic polymer, or a combination of the two fused together; formed using 3D printing or vacuum-forming; configured to engage all or part of the bottom and top teeth such that the lower jaw is advanced approximately 8 mm anterior to its habitual resting position and opened 8 mm from its habitual resting position when the appliance is applied; and configured such that 90% or more of airflow through the mouth is blocked by the appliance. In some embodiments, the appliance is configured to include gel-wells in the interior of the appliance.

In some embodiments, the appliance is further configured to connect with a CPAP hose.

In some embodiments, the appliance is further configured to firmly engage the undercuts of the teeth and inter-proximal spaces.

In some embodiments, the appliance the engagement of the undercuts of the teeth and inter-proximal spaces is such that suction is generated upon removing motion of the appliance.

### BRIEF DESCRIPTION OF DRAWINGS

FIG. 1 is a schematic illustration of a side view of a mouth showing upper and lower teeth in habitual rest position.

FIG. 2 is a schematic illustration of a side view of a mouth showing upper and lower teeth with lower teeth advanced relative to and separated from the upper teeth.

FIG. 3 is a schematic illustration of a side view of a mouth showing upper and lower teeth held by an example embodiment in a position that discourages snoring.

FIG. 4 is a schematic illustration of a side view of a mouth showing an example embodiment configured to accommodate a CPAP instrument.

FIG. 5 is a schematic illustration of an example embodiment of the present invention.

### MODES FOR CARRYING OUT THE INVENTION AND INDUSTRIAL APPLICABILITY

Embodiments of the present invention provide new, custom-made oral hybrid appliances (also referred to herein as an OHA), that can address one or more of the following needs during sleep:

(1) it is an Anti-Snoring Guard;

(2) it is an Anti-Teeth-Grinding NightGuard;

(3) it can be a very effective vehicle to deliver medicinal gels (e.g., fight against DryMouth), whitening gels and others.

Embodiments of the present invention can provide an improved mouth guard for sports and similar activities—the airway management can increase oxygen intake by 20% as compared with conventional sports appliances. Embodiments of the present invention can provide an appliance useful in treating mild to moderate sleep apnea—many patients with mild to moderate sleep apnea are mouth breathers during sleep.

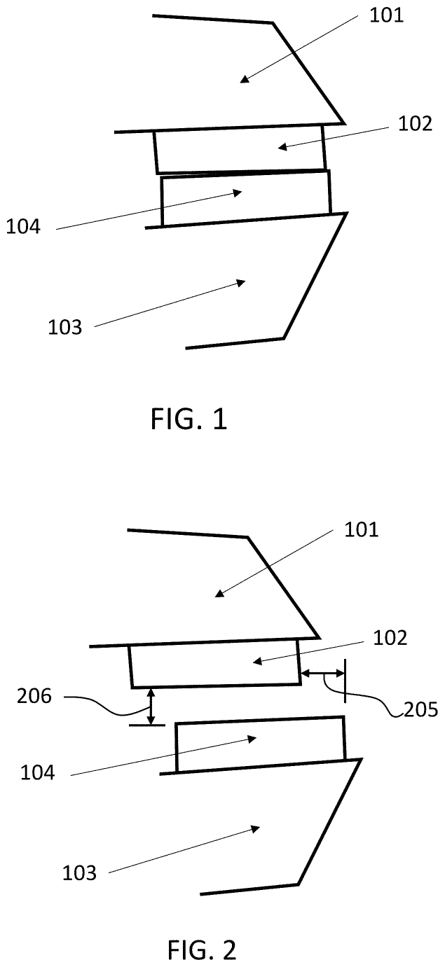

FIG. 1 is a schematic illustration of a side view of a mouth showing upper jaw 101 and teeth 102 and lower jaw 103 and teeth 104 in habitual rest position.

FIG. 2 is a schematic illustration of a side view of a mouth showing upper jaw 101 and teeth 102 and lower jaw 103 and teeth 104 with lower teeth advanced relative to and separated from the upper teeth. The lower jaw and teeth can be advanced relative to their habitual rest position, or relative to the upper jaw and teeth a predetermined distance 205, e.g., between 2 mm and 10 mm, between 6 mm and 10 mm, or 8 mm. The lower jaw and teeth can be separated relative to the upper jaw and teeth a predetermined distance 206, e.g., between 2 mm and 10 mm, between 6 mm and 10 mm, or 8 mm. The predetermined distances can also be determined relative to the needs or capabilities of a particular patient.

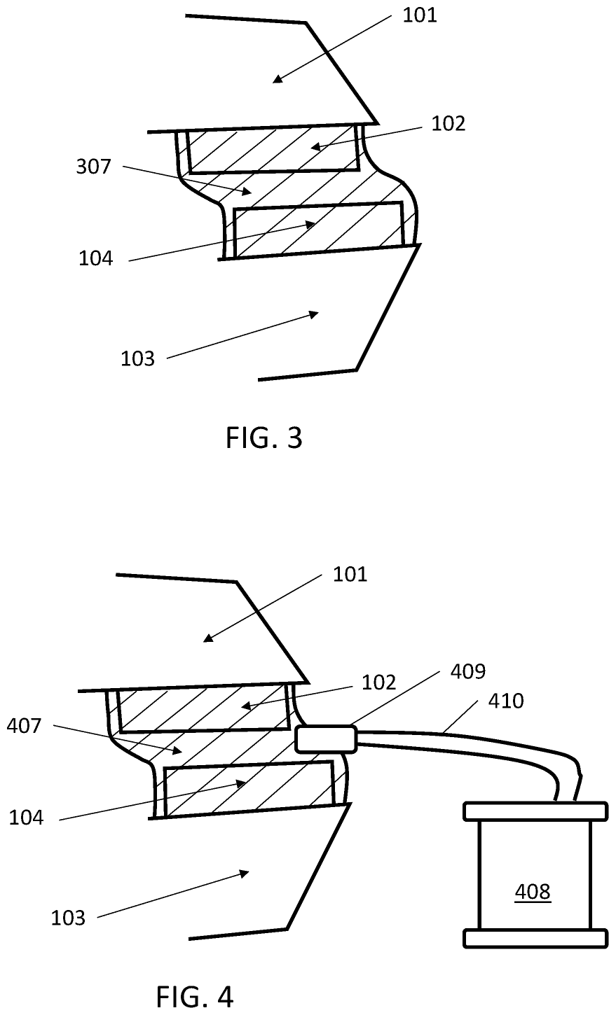

FIG. 3 is a schematic illustration of a side view of a mouth showing upper jaw 101 and teeth 102 and lower jaw 103 and teeth 104 held by an example embodiment appliance 307 in a position that discourages snoring. E.g., the lower jaw and teeth can be advanced relative to their habitual rest position by a distance, e.g., 8 mm. The lower jaw and teeth can be separated relative to the upper jaw and teeth by a distance e.g., 8 mm.

FIG. 4 is a schematic illustration of a side view of a mouth showing an example embodiment appliance 417 configured to accommodate a CPAP instrument 408. The example embodiment appliance 407 holds upper jaw 101 and teeth 102 and lower jaw 103 and teeth 104 in a position that discourages snoring. E.g., the lower jaw and teeth can be advanced relative to their habitual rest position by a distance, e.g., 8 mm. The example embodiment appliance 407 comprises an interface 409 that connects with air passage 410 (e.g., flexible tubing) to the CPAP instrument 408. Air passage 410 communicates forced airflow from the CPAP instrument 408 to the patient via interface 409.

### MATERIAL

Appliances according to the present invention can be made from 3D printable material or from sheaths using a vacuum suck down method. The materials used can be all elastomeric polymer or all hard non-elastic polymer or a combination of the two different materials fused together to ensure maximum comfort and durability, similar to athletic guards worn by professional and amateur athletes and Anti-Teeth-Grinding Night Guard, soft on the inside next to teeth surfaces for comfort and memory retention and hard on the outside for wear resistance

Softness of material, next to hard teeth surfaces and soft gingival tissues minimizes irritation and maximizes retention by easily engaging undercut hard tissue structures of the mouth when seated completely over the teeth.

The soft polymer, contacting hard teeth structures allows for a degree of desirable movement (not rigid like traditional custom made appliances) in all directions without disengaging from the teeth and resulting in loss of retention. This unique elastomeric property, prevents jaw soreness and significantly improves wear comfort.

### DESIGN

Example appliances according to the present invention can be designed to engage all or part of the bottom and top teeth in a very specific manner. Because the appliance is custom made from durable, elastomeric material with approximately 100% memory, the appliance easily and firmly engages the undercuts of the teeth and inter-proximal spaces to impart very comfortable retention to the upper and lower teeth during sleep.

Another unique feature of this material and design is its ability to add additional retention by its suction property. This additional retention through suction is not possible with traditional rigid plastics used in the fabrication of traditional appliances.

Another unique aspect of this appliance entails the specific positioning of the lower jaw relative to upper jaw. Clinical observation of appliance function indicates the lower jaw must be advanced an appropriate distance (e.g., approximately 8 mm in some examples) anterior to its habitual resting position and opened an appropriate distance (e.g., approximately 8 mm in some examples) from its habitual resting position. This very specific positioning of the lower jaw relative to the upper jaw can be critical to maintaining the airway sufficiently open to significantly increase air intake.

To stop snoring, it can be important that the patient not breathe through the mouth during sleep. Breathing through the mouth during sleep and not through the nose (as it should be normally at rest such as in during sleep) will result in the drying out of airway soft tissue membranes, producing laboured and uneven airflow over these dry membranes, resulting in the unwanted noxious snoring sound.

This unique appliance design blocks most air flow through the mouth (more than 90%), forcing the patient to breathe through the nose as it should be at rest, eliminating the snoring sound. Therefore, the design of this novel appliance incorporates no breathing holes and/or vents. Traditional appliances allow a very significant amount of airflow through the mouth via incorporated holes, vents and spaces between the upper and lower teeth creating unwanted whistling noises and significant amount of unwanted drooling. Drooling of saliva causes sore painful lesions in the corners of the mouth (Angular Chelitis). This appliance is contraindicated if the patient has nasal obstruction such as a cold, deviated septum or nasal polyps.

Another important and unique characteristic for this type of appliance is the strategic incorporation of gel-wells within its interior, to enable the delivery of medical agent gels and dental whitening gels to the teeth and gums if necessary.

Because this appliance, when worn does not allow the contact between the upper and lower teeth during sleep, it is also a very effective Anti-teeth grinding appliance.

The novel appliance can also be designed if desired to accommodate a CPAP air input hose. This novel design is less cumbersome, more comfortable and does not irritate facial structures.

### EXAMPLE EMBODIMENT

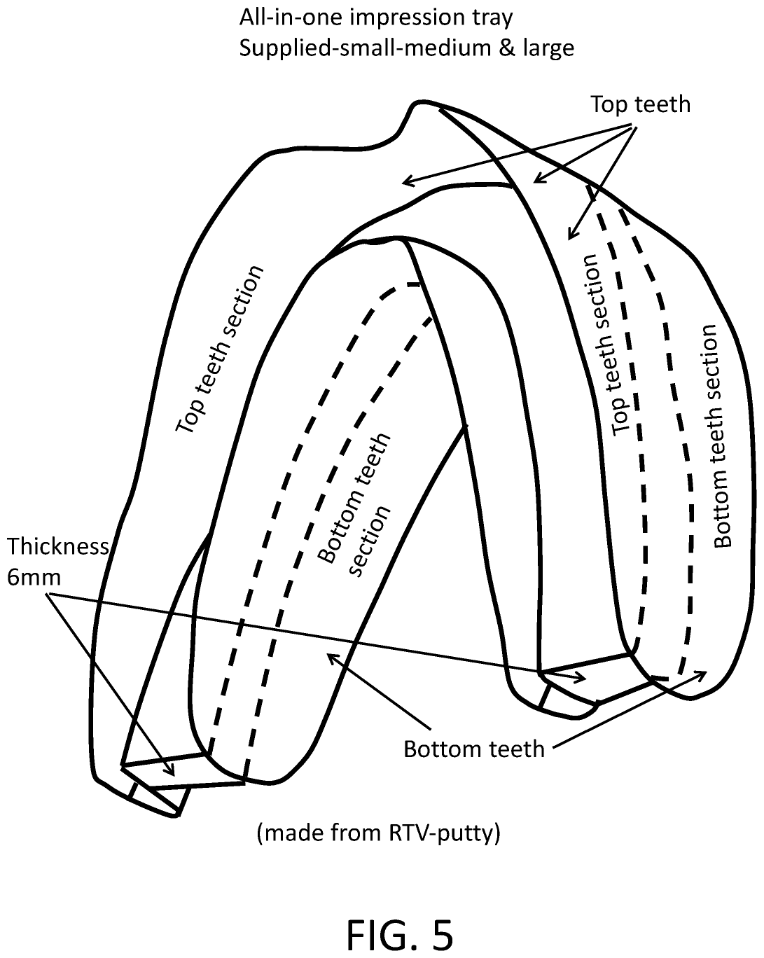

FIG. 5 is a schematic illustration of an example embodiment of the present invention. The embodiment provides an all-in-one impression tray, supplied in one of several sizes to accommodate various patients (e.g., small, medium, or large). The impression tray can be made of RTV silicone, and comprise a top teeth section and a bottom teeth section. The top teeth section and bottom teeth section can be spaced apart by an appropriate distance, e.g., 6 mm-8 mm in various examples.

The impression tray can be relined with soft RTV silicone putty. The patient bites into the putty, and holds the bite position until the putty is appropriately cured, e.g., about 3 minutes in some applications, depending on the putty material and operating conditions. Since the prefabricated impression tray and the putty used for customizing are the same material, the physical and chemical properties are the same, providing a lasting and durable union of the two.

An oral appliance according to the example embodiment can provide an anti-snoring appliance. It performs this function by (a) advancing (6 to 8 mm) and separating the lower jaw (6 to 8 mm) relative to the upper jaw and thereby opening up the posterior airway. It also blocks airflow through mouth by forcing airflow through the nose, as it normally should be. If there is no airflow through the mouth, there is no snoring. Snoring sounds are only produced via airflow through the mouth.

An oral appliance according to the example embodiment can also provide an anti-teeth-grinding appliance. If the teeth are kept apart, it is impossible to grind teeth.

An oral appliance according to the example embodiment can also provide an extremely effective cellular oxygenating device. Research suggests that breathing through the mouth while asleep will result in approximately 20% oxygen deprivation when compared to normal nasal breathing.

### Example Methods in connection with the Example Embodiment of FIG. 5

A patient can supply images of the patient's bite, e.g., by taking photos as they bite into a piece of bread, with a coin placed within the bite perimeter. The images can be transmitted to a dentist or other advisor, or an appropriate software program. The images can be used to determine an appropriate prefabricated oral appliance, e.g., sized to the patient to facilitate successful customization. The prefabricated appliance can be selected from a standard inventory, or can be produced on demand, e.g., by 3D printing, in a standard size or according to a template or algorithm based on the photos from the patient. The prefabricated appliance can be available in a number of sizes and configurations, from a single size to many. More sizes and configurations allow more control over the final appliance since the putty customizes to the patient's bite—the desired positioning is accomplished primarily by the prefabricated appliance. Thus it can be desirable to have a prefabricated appliance that is already a close match to the patient's mouth so that the desired positioning is obtained, and the putty only needs to customize the fit to the patient's individual tooth placement. Suitable prefabricated appliances can be realized by 3D printing as well as silicone injection and casting forming methods.

The selected prefabricated appliance can be delivered to the patient. Soft RTV silicone putty can be applied to the appliance. The patient then places the appliance in the mouth and bites into the putty. The patient holds the bite position until the putty is adequately cured, e.g., about 3 minutes for some currently available RTV silicone putties.

The patient can also supply additional patient-specific information, along with the photos, to a remote dentist. The dentist can confirm patient need and the appliance, and facilitate insurance claim for the cost.

The RTV silicone used for the appliance (prefabricated portion and the customization putty) can have a Shore Hardness property between 45 and 65. Hardness under 45 can be too soft to be useful as an oral appliance, while hardness above 65 can be too hard to be comfortable for the patient.

### Example Method

A method of producing a multi-functional oral appliance can comprise digital data input and manipulation (with minimal touch engineering) leading to the automatic 3D printing of the appliance. The process begins with the patient, from the comfort of their home, providing analog (silicone or other impressing materials) dental impressions of their teeth. These analog records can be mailed by the patient to a central collection center and converted into a 3D digital file, using a tabletop 3D scanner. As an alternative, if the patient has access to suitable technology, the patient can directly scan their teeth and upload the digital scan file.

Utilizing suitable software, the 3D digital data of the patient's teeth is fed into a digital pipeline and the end result is a multipurpose, custom made oral appliance. The appliance is prescribed by a teledentist (by analyzing the digital data somewhere along this pipeline) who does not need to see the patient personally. The prescription can be generated by the teledentist, solely based on the analysis of the data and not the physical patient. This can allow a patient to acquire a custom-made oral appliance with a much reduced professional fee and submit the appliance cost to his /her dental insurance for reimbursement.

The patient can wear the appliance as directed for a period, e.g., one or two weeks. The patient can make notes, or answer questions in a questionnaire, or otherwise provide information regarding fit, function, comfort, effectiveness, or other aspects of usage of the appliance. The information can be provided to a teledentist or other professional along with the actual appliance. The teledentist or other professional can trim or tailor the appliance to provide an improved user experience based on the information. A digital representation of the appliance can be determined, e.g., by the patient or by the teledentist or other professional, and the trimming or tailoring can be performed using the digital representation. The modified digital representation can be used to trim or tailor the actual appliance, or can be used to produce a replacement appliance, in each case providing the patient an improved appliance.

A replacement appliance can be fabricated using injectable silicon liquid (ISL), which can be more durable than RTV. ISL can be used to line or customize a standard appliance form, using 3D models of the patient's teeth, an appliance customized using RTV, information from patient feedback, or any combination thereof.

The present invention has been described in connection with various example embodiments. It will be understood that the above descriptions are merely illustrative of the applications of the principles of the present invention, the scope of which is to be determined by the claims viewed in light of the specification. Other variants and modifications of the invention will be apparent to those skilled in the art.

What is claimed is:

1. A method of producing a multi-functional oral appliance, comprising accepting images representative of a patient's oral characteristics; producing or selecting an appliance form based on the characteristics, where the form comprises a cured material; applying uncured material to the form; and causing the patient to bite into the form/putty combination and hold the bite position until the putty has cured;
wherein the form is configured such that the patient's top teeth are positioned in a predetermined relationship relative to the patient's bottom teeth when the patient bites into the form,
wherein the predetermined relationship comprises the bottom teeth shifted (a) forward a first distance relative to the top teeth, (b) away a second distance from the top teeth, or (c) both, relative to the patient's normal resting position;
wherein the distance is not less than 2 mm and not more than 10 mm

2. The method of claim 1, wherein the bottom teeth are shifted forward relative to the top teeth.

3. The method of claim 2, wherein the distance is between 4 mm and 8 mm.

4. The method of claim 1, wherein the bottom teeth are shifted away from the top teeth.

5. The method of claim 4, wherein the distance is between 4 mm and 8 mm.

6. The method of claim 1, wherein the bottom teeth are shifted forward a first distance relative to the top teeth, and shifted a second distance away from the top teeth, relative to the patient's normal resting position.

7. The method of claim 6, wherein the first distance is not less than 2 mm and not more than 10 mm.

8. The method of claim 6, wherein the second distance is not less than 2 mm and not more than 10 mm.

9. The method of claim 7, wherein the first distance is between 4 mm and 8 mm.

10. The method of claim 8, wherein the second distance is between 4 mm and 8 mm.

11. The method of claim 1, further comprising collecting information concerning the patient's experience in use of the appliance, then modifying the appliance responsive to such information.

12. The method of claim 1, further comprising collecting information concerning the patient's experience in use of the appliance, and determining a digital representation of the appliance, then producing a second appliance responsive to the information and the digital representation.

12. The method of claim 1, wherein the form is selected by a teledentist.

14. A multi-function oral appliance, comprising an elastomeric polymer, a hard non-elastic polymer, or a combination of the two fused together; formed using 3D printing or vacuum-forming; configured to engage all or part of the bottom and top teeth such that the lower jaw is advanced approximately 8 mm anterior to its habitual resting position and opened between 2 mm and 10 mm from its habitual resting position when the appliance is applied; and configured such that 90% or more of airflow through the mouth is blocked by the appliance; and configured to include gel-wells in the interior of the appliance configured to retain gel adjacent a tooth, soft tissue, or both.

15. The multi-function oral appliance of claim 14, further configured to connect with a CPAP hose.

16. The multi-function oral appliance of claim 14, wherein the appliance is configured to firmly engage the undercuts of the teeth and inter-proximal spaces.

17. The multi-function oral appliance of claim 14, wherein the appliance engagement of the undercuts of the teeth and inter-proximal spaces is such that suction is generated upon removing motion of the appliance.
